(19) United States
(12) Patent Application Publication
(10) Pub. No.: US 2025/0035652 A1
(43) Pub. Date: Jan. 30, 2025

(54) DEVICES, SYSTEMS AND METHODS FOR DETECTION OF BETA SUBUNIT OF LUTEINIZING HORMONE

(71) Applicant: MFB Fertility, Inc., Erie, CO (US)

(72) Inventor: Amy BECKLEY, Erie, CO (US)

(73) Assignee: MFB Fertility, Inc., Erie, CO (US)

(21) Appl. No.: 18/696,892

(22) PCT Filed: Oct. 17, 2022
(86) PCT No.: PCT/US2022/046902

§ 371 Date: Mar. 28, 2024

Related U.S. Application Data
(60) Provisional application No. 63/256,987, filed on Oct. 18, 2021.

(51) Int. Cl. G01N 33/76; C07K 16/26; G01N 33/543

(52) U.S. Cl. CPC G01N 33/76; C07K 16/26; G01N 33/54388; G01N 2333/59

(57) ABSTRACT

Provided herein are urine-based lateral flow assays for the detection of the beta subunit of luteinizing hormone (LH), and methods of interpretation and digital quantification thereof. Such assays may be incorporated into ovulation test devices, e.g., for evaluating the onset and quality of ovulation. Also provided are diagnostic systems, and methods of using such devices and systems.

### CROSS REFERENCE TO RELATED APPLICATIONS

This application claims priority to U.S. Provisional Application Ser. No. 63/256,987, filed Oct. 18, 2021, which is hereby incorporated herein by reference in its entirety.

### FIELD

The present disclosure relates generally to hormone diagnostics, and more specifically to urine-based lateral flow assays for the detection of the beta subunit of luteinizing hormone (LH), and methods of interpretation and digital quantification thereof.

### BACKGROUND

Hormones circulate in the blood and act within the blood to produce varies results. However, not all hormones are produced in a consistent way. In fact, many of the reproductive hormones follow circadian patterns, and/or are subject to negative or positive feedback loops. The positive and negative feedback effects of estrogen and progesterone are essential in regulating the cyclical activity of the hypothalamic-pituitary-ovarian axis. When progesterone is high after ovulation, it inhibits gonadotrophin-releasing hormone (GnRH) and luteinizing hormone (LH) secretion. Conversely, when progesterone is low, GnRH/LH act to increase progesterone by inducing a pulse of progesterone from the ovary the ovum was ovulated from (He et. al, 2017).

Progesterone can block the estradiol-induced GnRH/LH surge and inhibit LH pulse frequency. Recent studies reported that progesterone prevented premature LH surges during ovarian hyperstimulation in women. As the most potent stimulator of GnRH/LH release, kisspeptin is believed to mediate the positive and negative feedback effects of estradiol in the hypothalamic anteroventral periventricular (AVPV) and arcuate (ARC) nuclei, while the region-specific role of progesterone receptors in these nuclei remains unknown. Clinically, premature spontaneous LH surges are a major cause of cycle cancellation in women and recently, progesterone has been shown to successfully prevent premature LH surges in women undergoing ovarian stimulation, thereby validating its use in in vitro fertilization (IVF) regimes. Despite its clinical and physiologic importance, the neural mechanisms underlying the inhibitory actions of progesterone on surge, and indeed pulsatile, release of GnRH/LH remain poorly understood. While it is understood that the release of progesterone does have an inhibitory action on LH pulse frequency and the production of LH. Thus, an improved system to better detect and understand the various forms of LH production, particularly in the context of the detection and understanding of the presence of progesterone in a woman's body, remains desirable (Rahman et. al. 2019).

Serum hormone levels fluctuate, causing a single blood test analysis to have poor diagnostic value for assessing ovarian or ovulation function. Female reproductive hormones fluctuate within a single day in the blood, but are not usually considered when interpreting test results. Estrogen, progesterone, follicle-stimulating hormone (FSH) and luteinizing hormone (LH) show significant 24-hour rhythms during the menstrual cycle. In other words, female reproductive hormones are under endogenous circadian regulation. The hormonal peaks have been found to occur in the morning for progesterone, in the afternoon for FSH and LH, and during the night for estrogen. Thus, there are benefits of measuring urine hormones as opposed to blood levels, besides being much more convenient and painless. Therefore, an improved system to better detect and understand the various forms of urine hormones and analytes associated with ovarian and ovulation function remains desirable.

The levels of hormones generally associated with a menstrual cycle have a tendency to exhibit different levels at different times in a woman's menstrual cycle. For example, Estrogen levels are low in the morning and highest at night. A single blood draw, therefore, might not be an accurate measure of the average level of estrogen circulating in the body. Also, because estrogen levels fluctuate day to day during the menstrual cycle, it is preferable to obtain multiple days of testing results to determine trends over a single menstrual cycle or from one cycle to another cycle. Estrogen is metabolized to estrone-3-glucuronide (also known in the art as E1G or E3G) in the liver and secreted into the urine. Therefore, because productive hormones are metabolized the most while you sleep, E1G measures are best taken using the first urine after your longest sleep. A rise in E1G detectible in urine provides an early indication for the opening of the fertile window during a woman's menstrual cycle.

Progesterone levels in the serum vary as much as eight-fold (+−800%) over a 16-hour period. Typically, progesterone levels are highest in the morning fall during the day, then increase at night again. Again, that is why a single blood draw is not an accurate measure of progesterone levels. Progesterone is metabolized into pregnanediol glucuronide (PDG) in the liver and secreted into the urine. Like estrogen, this metabolism occurs mainly while you sleep, making the urine after your longest sleep the most accurate for measuring PDG levels.

Unlike estrogen and progesterone, which are steroid hormones, luteinizing hormone (LH) is a heterodimeric glycoprotein that includes one alpha and one beta subunit make the full, functional protein. The LH hormone is a member of the glycoprotein hormone family (GPH), which also includes human chorionic gonadotropin (HCG), follicle stimulating hormone (FSH), and thyroid stimulating hormone (TSH). All members of the GPH family are heterodimers and includes an alpha and beta subunit. The alpha subunit is conserved across the GPHs, while the beta subunit is unique. The beta subunits share about 80% homology across the GPH members.

Intact LH refers to the combination of an alpha subunit of LH and a beta subunit of LH. Intact LH circulates in the bloodstream and filters into the kidneys. Once in the kidney, Intact LH will degrade, and following that process only LH-beta subunits will remain. Urine can contain both intact LH and LH-beta. In assays configured to detect for the presence of or otherwise measure for Intact LH, both the alpha subunit of LH and the beta subunit of LH must be present in the fluid applied to the assay to generate the result. Most commercially available LH tests measure intact LH, including the present applicant's commercially available “Predict LH” tests. Prior art LH tests measure intact LH alpha/beta together. In such examples, one antibody measures the LH alpha subunit and the other antibody measures the beta subunit, so Intact (or “total”) LH must be secreted into the urine as a whole to be detected or measured by such prior art ovulation tests.

However, benefits would be gained by instead measuring only the beta subunit of LH on a standalone basis. The beta subunit of LH (LH beta) exhibits unique characteristics that can be specifically useful in association with the diagnosis of conditions associated with fertility, menopause and other conditions associated with women's health. For example, in addition to conferring differentiation, the beta subunit also grants each heterodimer its unique biological activity and receptor specificity. Thus, an alternative system specifically configured to detect for and/or measure the presence of LH beta in urine remains desirable.

Part of the benefit of testing LH beta separately from Intact LH derives from the timing within the menstrual cycle that they appear. For example, it is well known in the art that intact LH surges in the urine of women about 24 hours prior to ovulation. The intact LH will remain elevated for 4-48 hours then drop back down. Academic literature also demonstrates that in about 20% of women, the surge in urine intact LH occurs after ovulation. As most prior art LH tests purport to identify the onset of ovulation and the opening of the fertile window, the fact that such tests only are configured to detect Intact LH, remains a problem causing inaccurate indications. An improvement therefore remains desirable.

LH beta levels, on the other hand, typically begin to increase up to 3 days prior to ovulation, in contrast to Intact LH, which typically increases only 1 day before ovulation. Thus, LH beta could give some women an earlier indication of their fertile window, better helping them time intercourse. Moreover, LH beta levels remain high for an average of 4-5 days while intact LH typically remains elevated 1 or 2 days. Therefore, women with short surges might have a better window to detect their surge if they measure LH beta on a standalone basis. Therefore, an improved system or device configured to measure LH beta on an isolated basis, as opposed to Intact LH, remains desirable.

Thus, there remains a need for a urine-based test that can selectively or preferentially detect LH beta. There also remains a need for an ovulation test device exhibiting an improved level of accuracy for determining ovulation status, the onset of ovulation or the opening of the fertile window.

### BRIEF SUMMARY

In some aspects, provided herein are devices, systems and methods that address at least some of the aforementioned needs by providing a test that can selectively or preferentially detect LH beta. In some embodiments, provided is a device for selectively or preferentially detecting LH beta in a non-serum bodily fluid deposited on a proximal portion of the device for transport to a distal portion of the device. The device includes a conjugate pad formed of a first material having a detectable label thereon and a membrane in fluid communication with the conjugate pad and formed of a second, different material. The membrane includes a test line. At least one of the conjugate pad and the membrane includes a binding member that exhibits a moderate to high affinity for LH beta and is selectively or preferentially reactive with LH beta. The devices provided herein can allow the user to predict ovulation or the opening of the fertile window, as LH beta provides an early indication of a fertile window, and LH beta can be detected by the assay.

In other embodiments, provided is a device for selectively or preferentially detecting LH beta, in which the device includes a conjugate pad formed of a first material and having a detectable label thereon. The device also includes a membrane in fluid communication with the conjugate pad and formed of a second (and/or a third, fourth, etc.) different material. The membrane includes a test line. In some variations, the device further includes a second, third or fourth test line that is selectively or preferentially reactive with another hormone or analyte. Hormones or analytes may include, progesterone, estrogen (including, for example, estrone, estradiol and estriol), follicular stimulating hormone (FSH), or any metabolites of the foregoing. In one variation, the hormones or analytes may include progesterone glucuronide (PDG). In another variation, the hormones or analytes may include estrone-3 glucuronide (E1G or E3G). In another variation, the hormones or analytes may include Intact LH. The additional test lines can be located anywhere between the conjugate pad and end of the detection zone. Further, at least one of the conjugate pad and the membrane can include a binding member that is reactive with LH beta. In one embodiment, the binding member can be selectively or preferentially reactive with LH beta and also exhibit a moderate to high affinity for LH beta.

In certain embodiments, the device for selectively or preferentially detecting LH beta in a non-serum bodily fluid deposited on a proximal portion of the device for transport to a distal portion of the device includes a conjugate pad formed of a first material and having a detectable label and a membrane in fluid communication with the conjugate pad formed of a second, different material. The membrane includes a test line. Additionally, such devices include a mixture of binding members. In these embodiments, the mixture of binding members includes a first group of binding members that are selectively or preferentially reactive with LH beta, or any part of LH beta, or LH alpha, or any part of LH alpha.

In some embodiments, the indication by the test strip of the onset of ovulation is presented by a detectable label. In certain embodiments the detectable label is perceptible by the human eye. In other embodiments, the detectable label includes fluorescent dye imperceptible to the human eye readable only with the assistance of a base unit or digital reader. The immunoassay devices and methods may be used in conjunction with diagnostic reader systems and/or a base unit for obtaining a sensitive read-out of the immunoassay results.

In another aspect, provided is a method of evaluating the onset and quality of ovulation. Methods described herein include providing a test device for selectively or preferentially detecting LH beta, as described herein, and applying a non-serum bodily fluid potentially including one or both of LH beta and another hormone or analyte, potentially PDG, to the device. The detected presence of LH beta indicates the onset of ovulation, whereby the detected presence of PDG on at least two of the days during the period of 7-10 days following the detected presence of LH beta indicates a high quality of ovulation.

Configurations of immunoassay devices and methods disclosed herein may include those described further in U.S. Pat. Nos. 11,029,321; 11,061,026, and 11,131,665; U.S. patent application Ser. Nos. 15/900,794; 15/974,229; 16/381,229; 16/544,554; 17/317,212; 17/446,369; 62/460,307; 62/503,223; 62/611,467; 62/720,953; 63/023,116; and 63/112,051; PCT Application Nos. PCT/US18/68027 and PCT/US2020/040600, each of which is incorporated by reference in its entirety herein.

### DESCRIPTION OF THE FIGURES

The present application can be understood by reference to the following description taken in conjunction with the accompanying figures.

FIG. 1 depicts an exemplary method to estimate the opening of the fertile window.

FIG. 2 depicts an exemplary method to determine the onset of ovulation and evaluate the quality of ovulation.

FIG. 3 depicts an exemplary system using an application operating on a mobile device featuring a camera.

FIG. 4 depicts an exemplary test strip featuring a control line and a test line for detecting the presence or absence of LH beta.

FIGS. 5A and 5B depict embodiments of an exemplary base unit shown in an intended use in conjugation with a cartridge containing a test trip configured to evaluate a bodily fluid (e.g., urine sample) for the presence or absence of LH beta.

FIG. 6 depicts an embodiment of a test strip contained within a cartridge in exemplary use in association with a base unit.

FIG. 7 depicts an exemplary test strip that detected presence of LH beta, estrogen metabolite E1G and progesterone metabolite PDG in a single urine sample.

FIG. 8 depicts a comparison using an assay that detects LH beta with an assay that detects Intact BH to determine LH surge at a threshold.

### DETAILED DESCRIPTION

The following description sets forth exemplary methods, parameters and the like. It should be recognized, however, that such description is not intended as a limitation on the scope of the present disclosure but is instead provided as a description of exemplary embodiments.

In some aspects, provided herein are devices, systems and methods for evaluating the onset of ovulation and/or quality of ovulation. The devices, systems and methods may be used for evaluating a non-serum bodily fluid daily over a period of multiple days for the presence of at least LH beta beyond a threshold to detect for the onset of ovulation. The devices, systems and methods employ lateral flow assays configured to detect LH beta.

In certain aspects, provided herein is an ovulation test device. The device includes a proximal portion in fluid communication with a distal portion. A non-serum bodily fluid (e.g., urine) can be directly or indirectly deposited on the proximal portion of the device for transport to the distal portion. In general, embodiments include a first binding member located on the proximal portion of the device and a second binding member deposited on the distal portion of the device. In some variations, at least one of the binding members exhibits a moderate to high affinity for LH beta while also being selectively or preferentially reactive with LH beta.

In some variations, a “moderate to high affinity” antibody or binding member thereof comprises an antibody or binding member that can bind with a particular antigen within a relatively short incubation time. Likewise, an antibody or binding member thereof “exhibits a moderate to high affinity” for a particular antigen should be understood as requiring a relatively short sample incubation time. For instance, an antibody or binding member exhibits “moderate to high affinity” at equilibrium (“KA”) greater than 1e⁹ (e.g., 1e⁹ to 1e¹¹, 1e⁹ to 1e¹⁰, or 1e¹⁰ to 1e¹¹) or preferably at least 1e¹⁰ (e.g., 11e¹⁰ to 1e¹²). In certain embodiments, KA values at equilibrium between about 1e⁹ to 1e¹⁰ can be deemed as exhibiting a moderate affinity, while KA values at equilibrium of at least 1e¹⁰ can be deemed as exhibiting a high affinity. At equilibrium, KA values of 1e⁸ or less can be deemed as exhibiting a low affinity. Such KA values can be experimentally determined using any suitable method known in the art.

Such devices also include a test line located on the distal portion of the device that directly or indirectly binds the LH beta. The presence of LH beta in the non-serum bodily fluid can be determined by visual inspection of the test line, where the presence of LH beta is indicated by the presence of color development at the test line. Accordingly, embodiments provide a device that selectively or preferentially detects LH beta.

Generally, an antibody is selectively reactive with a protein or epitope thereof when the antibody functions in a binding reaction with the protein without significantly binding with other proteins. In some variations, an antibody is selectively reactive with LH beta if the antibody exhibits greater than 50% differential discrimination of LH beta over Intact LH. In certain variations, an antibody is preferentially reactive with LH beta if the antibody exhibits greater than 60%, 70%, 80, or 90% differential discrimination of LH beta over Intact LH. Similarly, an antibody is preferentially reactive with Intact LH if the antibody exhibits greater than 50% differential discrimination of Intact LH over LH beta. In certain variations, an antibody is preferentially reactive with Intact LH if the antibody exhibits greater than 60%, 70%, 80, or 90% differential discrimination of Intact LH over LH beta.

In some embodiments, the device specifically differentiates between LH beta and Intact LH. As used herein, “Intact LH” includes both LH alpha and LH beta subunits.

In some aspects, provided herein is a method of evaluating the onset of ovulation, comprising: providing any of the devices described herein for selectively or preferentially detecting LH beta; applying a non-serum bodily fluid comprising (i) LH beta or (ii) LH beta and another hormone or analyte to the device; and detecting the presence of LH beta, wherein the detected presence of LH beta indicates the onset of ovulation.

The devices, systems and methods provided herein use non-serum bodily fluids to evaluate the onset of ovulation and quality of ovulation. In some embodiments, the non-serum bodily fluid is urine. In some variations, to effectively facilitate the method when the non-serum bodily fluid tested is urine, a sample is collected immediately after the woman whose corpus luteum is under evaluation wakes up from the longest sleep period of the preceding twenty four hours. The sample from the first morning urine may present a more accurate representation of the previous day's serum LH beta levels.

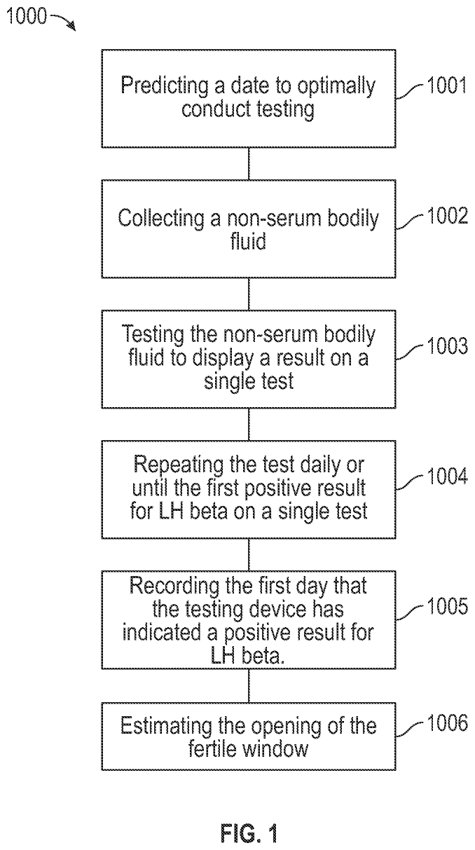

With reference to FIG. 1, exemplary method 1000 to evaluate the onset of ovulation utilizing specialized testing devices, optionally single tests, is depicted. Method 1000 includes a step 1001 of predicting a date to optimally conduct testing. In an example, the ovulation date is first estimated. During this step, the ovulation date is estimated by sampling cervical mucus, retrieving a basal body temperature, or counting days from the date of beginning menstruation. In some embodiments, the estimated ovulation date is utilized to calculate the optimal date or range of dates to perform a test to evaluate a bodily fluid for the presence or absence of any of LH beta, progesterone or progesterone metabolites, PDG, LH, HCG or Estrogen or estrogen metabolites. Optionally, the ovulation date is utilized to calculate the optimal date or range of dates to conduct bodily fluid testing. Then, following the predicting an ovulation date step, the method preferably includes a collecting step 1002. During step 1002, a non-serum bodily fluid sample daily is collected daily starting 3-15 days after the onset of menstruation. In the case where the non-serum bodily fluid collected is urine, the sample may be collected within a cup during urination. In some instances, where the testing device is configured to allow the urine sample to be collected mid-stream during urination, the urine sample may be collected directly onto the testing device. In some variations of the method where the non-serum bodily fluid collected is urine, a urine sample is taken in the morning during the first urination after the subject awakens from overnight sleep or the longest sleep of the day (referred to as “first morning urine”). Alternatively, in the case where the non-serum bodily fluid sample collected is saliva, the sample may be collected with a swab. The collection step takes place starting 3-15 days after the onset of menstruation. In any case, the objective of the step is to collect enough of a sample to allow for the non-serum bodily fluid to be temporarily held as needed, and then evaluated by a testing device.

Then, following the collecting step, a testing step 1003 takes place. During the testing step, for each non-serum bodily fluid sample, a testing device is used to evaluate the non-serum bodily fluid sample for the presence of LH beta. In one embodiment, step 1003 utilizes a single test that involves a single-use disposable non-serum bodily fluid test configured to detect for at least LH beta above a pre-defined threshold. In some embodiments, the non-serum bodily fluid test is configured to evaluate urine for the presence of LH beta, above a pre-defined threshold of 15 mIU/mL. In one variation, the pre-defined threshold is above 20 mIU/mL. In some embodiments, the pre-defined threshold is determined from a value chosen from a sliding scale. In some embodiments, a threshold from within the scale's range is a value in a range between 15-45 mIU/mL. In one variation, a threshold from within the scale's range is a value in a range between 20-25 mIU/mL.

It should also be understood that reference to “between” two values or parameters herein includes (and describes) embodiments that include those two values or parameters per se. For example, description referring to “between x and y” includes description of “x” and “y” per se.

During the testing step, the single test indicates whether LH beta is present in the tested non-serum bodily fluid sample above a pre-defined threshold. A result indicated by the single test of “positive” means that the level of LH beta present in the tested non-serum bodily fluid sample has exceeded the pre-defined threshold. A result indicated by the single test of “negative” means that the level of LH beta present in the tested non-serum bodily fluid sample has not exceeded the pre-defined threshold. In a related example, a result indicated by the single test of “positive” could further indicate the opening of the fertile window and onset of ovulation.

Then, following the first positive result indicated by the single test, indicating the LH beta above a pre-defined threshold in the tested non-serum bodily fluid sample, or the first fold change, a repeating step 1004 takes place.

In method 1000, repeating step 1004, collecting step 1002 and testing step 1003 may be performed over and over on a daily basis until the first positive result for LH beta on the single test. When LH beta is above the pre-defined threshold, a positive result for LH beta is observed, which indicates that the fertile window has opened and that a woman's most fertile timeframe during her menstrual cycle is imminent. In other words, the first positive result for LH beta is an indicator of the imminency of the woman's peak fertile day. Prior to the first positive result for LH beta, contemporaneous testing to detect the presence of E1G above a threshold or a fold change rise in E1G may present an earlier indication of the opening of the woman's fertile window during the menstrual cycle. Any suitable methods known in the art may be employed to test for E1G.

Following the first positive result for LH beta above a pre-defined threshold, PDG can be measured for 3 consecutive days, or for 4-10 days, or for the period of time between 7-10 days after first positive result for LH beta. Alternatively, repeating step 1004 takes place daily for only 3 consecutive days, or for 4-10 days, regardless of the results displayed on the testing device. In the event that a positive result for the presence of LH beta occurs at least two times during the repeating step, the onset of ovulation is indicated. The present inventor has recognized that performing repeating step 1004 multiple times during a fixed period of time, such as a timeframe chosen from the range of 3-10 days, occasionally has the effect of mitigating errors displayed on the single test. When using Intact LH tests, since intact LH can only last 4-6 hours, Intact LH surge might be missed with once a day testing. By testing once daily for LH beta, one can decrease the chances of missing an LH surge as, LH beta surge typically lasts 2-3 days or more. The enhanced sensitivity of using LH beta in daily LH testing surprisingly reduces the number of LH tests a subject (e.g., a woman) must perform to detect a LH surge. Thus, in some variations, the devices, systems and methods here can detect LH beta 2-3 days earlier than Intact LH, and LH beta and Intact LH can be detected up to the same date.

Following the conclusion of repeating step 1004 or multiple repeating steps 1004, where one or more additional iterations of the collecting step 1002 and the testing step 1003 have taken place, a recording step 1005 takes place.

During step 1005, the first day that the single test has indicated a positive result for LH beta above the pre-defined threshold at the time of testing, is recorded. In the example where a first single positive result for LH beta has been indicated, the onset of ovulation is indicated. Such recordation may optionally take place with the assistance of a calendar or similar application operating on a mobile device. Such application may optionally also allow its user to record results for testing devices that evaluate bodily fluids for progesterone or progesterone metabolites (such as PDG), Intact LH, follicle stimulating hormone (FSH), and/or estrogen or estrogen metabolites.

Following recording, step 1006 estimates the opening of the fertile window. In some embodiments, the earliest indication of the opening of the fertile window is provided by detection of a rise of estrogen metabolites, including for example E1G, above a threshold or a fold change rise of estrogen metabolites relative to earlier days of testing during the same menstrual cycle. As noted above, any suitable methods known in the art may be employed to test for E1G. In some embodiments, the opening of the fertile window may also be signaled by the first indication of LH beta above a pre-defined threshold. In some embodiments, the pre-defined threshold is a high value of LH beta. In some embodiments, the high value of LH beta is indicative of an LH beta surge. In some variations, a urinary LH beta concentration of 15-45 mIU/mL or 20-25 mIU/mL can be regarded as a universal threshold indicative of the LH beta surge. Once the LH beta surge has been detected, it can be said that a woman's most fertile day is imminent. Then, in some embodiments, testing for progesterone or progesterone metabolites (such as PDG) can confirm successful ovulation.

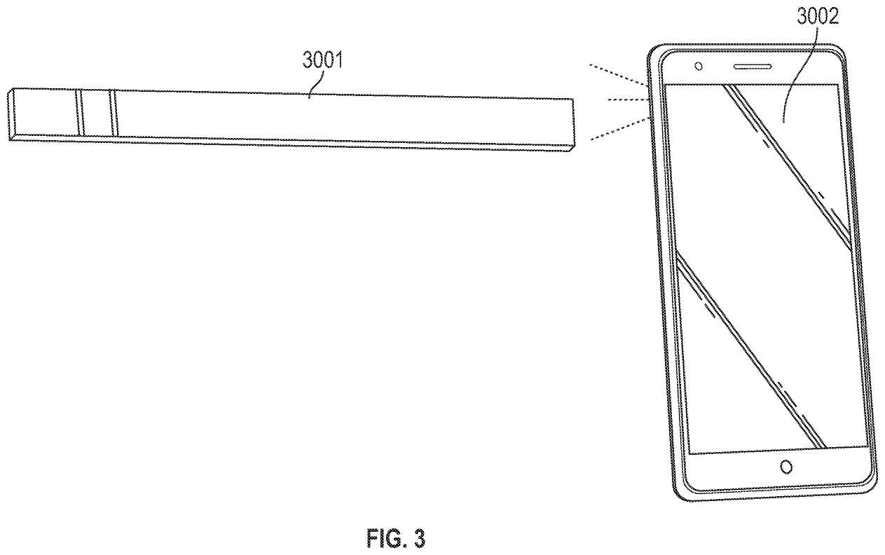

The present inventor has noted that a mobile device operating an application featuring the ability to facilitate step 1005 and/or step 1006 described above. In some embodiments, an application operating on a mobile device featuring a camera, as depicted in FIG. 3, may be configured and utilized to photograph the results of a single test, and near-simultaneously interpret and record the results of each single test. The present inventor has noted that this example of the method is particularly helpful in instances where a single test incorporates one or more test strips configured to evaluate for multiple hormones and/or analytes as is further described in U.S. patent application Ser. No. 16/381,229, which is incorporated by reference herein in its entirety. With reference to FIG. 3, test strip 3001 may be configured to evaluate for multiple hormones and/or analytes. In other embodiments, the test strip used in accordance with the methods described herein is configured to evaluate urine for the presence of PDG and LH beta. In certain aspects, the test results are interpreted with the assistance of a digital reader 3002. In an embodiment, the digital reader 3002 includes a mobile phone device as depicted in FIG. 3.

In employing the methods and systems described herein, it should be understood that, typically, E1G will open a woman's fertile window 2-4 days before the LH surge, which is the onset on ovulation. PDG confirms if ovulation happened, since sometimes LH does not actually result in ovulation. In certain aspects, provided herein is a method of evaluating the onset and quality of ovulation in a subject, comprising: providing any of the devices described herein for selectively or preferentially detecting LH beta; applying a non-serum bodily fluid comprising LH beta and PDG; detecting the presence of LH beta and PDG, wherein the detected presence of LH beta indicates (i) imminency of the subject's peak fertile day, and (ii) the onset of ovulation if PDG is also subsequently detected, and wherein the detected presence of PDG on at least two of the days during the period of 7-10 days following the detected presence of LH beta indicates a high quality of ovulation.

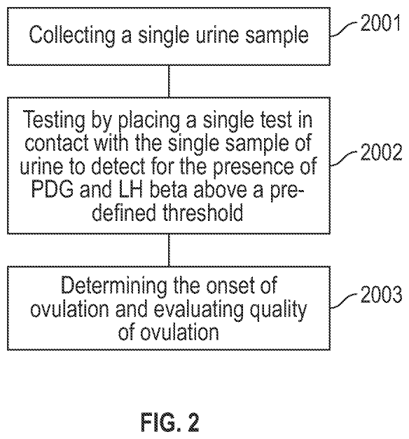

With reference to FIG. 2, exemplary method 2000 to evaluate the onset of ovulation and quality of ovulation is depicted. Step 2001 involves collecting a single sample of urine, which may involve a woman urinating into a cup. Then, a testing step 2002 follows. During the testing step, a single test configured to evaluate urine is placed into contact with the previously collected single sample of urine and utilized to detect for whether both PDG and LH beta are present in the single sample of urine, each above a pre-defined threshold.

Following testing step 2002, step 2003 determines the onset of ovulation and evaluates the quality of ovulation. An indication on a single test that LH beta exceeds a predefined threshold, optionally 15-45 mIU/mL or 20-25 mIU/mL, signals the onset of ovulation. Quality of ovulation is also determined by the detected presence of PDG on at least two of the days during the period of 7-10 days following the detected presence of LH beta which indicates a high quality of ovulation.

Following the determination of whether ovulation has occurred, the fertile window may also be determined. In some embodiments, estrogen metabolite increase is used to signal the opening of the fertile window and PDG increase in used to signal the close of the fertile window. In some embodiments, LH beta increase is used to signal the opening of the fertile window and PDG increase in used to signal the close of the fertile window. In some embodiments, LH beta increase is used to signal the most fertile day during a menstrual cycle and PDG increase in used to signal the close of the fertile window. During an example of such step, the fertile window is estimated by calculating a number of days, following the first positive indication of LH beta after a negative indication for LH beta from the immediately preceding day's testing. In some variations, a urinary LH beta concentration of 15-45 mIU/mL or 20-25 mIU/mL, can be regarded as a universal threshold indicative of the LH beta surge. Once the LH beta surge has been detected, it can be said that ovulation is imminent. Note, however, that a LH surge does not always result in ovulation.

An alternative method to self-confirm ovulation without hormone evaluation is through the tracking of basal body temperature (BBT). In association with such method, a female's temperature is tracked shortly after awaking in the morning on a daily basis. When the temperature significantly rises, by 0.5-1 degree fahrenheit or more, ovulation is indicated. The estimating the fertile window step also optionally comprises confirming a negative indication for PDG in urine, as a positive indication would signify that the fertile window has closed as the presence of PDG correlates to prior ovulation. A method for predicting the end of the fertile period is to measure the levels of the urinary hormone PDG. PDG has a relatively low level in urine until the start of the luteal phase, at which point its level rises fairly sharply. Therefore, once an elevated level of PDG is detected, it can be indicated to the user that the luteal phase of the cycle—i.e. the terminal infertile period—has commenced.

In some aspects, provided is a device for selectively or preferentially detecting LH beta in a non-serum bodily fluid deposited on a proximal portion of the device for transport to a distal portion of the device. In some embodiments, the device comprises: a conjugate pad formed of a first material having a detectable label thereon; and a membrane in fluid communication with the conjugate pad and formed of a second, different material, wherein the membrane comprises a test line. In some variations, at least one of the conjugate pad and the membrane comprises a binding member that exhibits a moderate to high affinity for LH beta, and is selectively or preferentially reactive with LH beta.

In some embodiments, the binding member is at least one antibody. In some variations, the antibody is a polypeptide substantially encoded by an immunoglobulin gene or immunoglobulin genes, or fragments thereof, which specifically recognize and bind an antigen. The recognized immunoglobulin genes include the kappa, lambda, alpha, gamma, delta, epsilon, and mu constant region genes, as well as the immunoglobulin variable region genes. In certain variations, antibodies include fragments, such as Fab′, F(ab)₂, Fabe, and Fv fragments. In certain variations, antibody includes antibody fragments either produced by the modification of whole antibodies or those synthesized de nova using recombinant DNA methodologies, and further includes humanized antibodies made by now conventional techniques.

In certain embodiments, the binding member is a binding site of the antibody that is selectively or preferentially reactive with an epitope of a hormone or analyte. In certain embodiments, such moderate to high affinity antibodies exhibit limited to minimal binding to Intact LH or other hormones or analytes that may be present in a non-serum bodily fluid. For instance, two mouse monoclonal antibodies (MMA's) have been developed which selectively or preferentially recognize and bind two distinct epitopes within LH Beta. Examples of antibodies that are selectively or preferentially reactive with LH Beta that specifically may be used in the test strip, systems and methods described herein include, for example, MLH-3 and MLH-6.

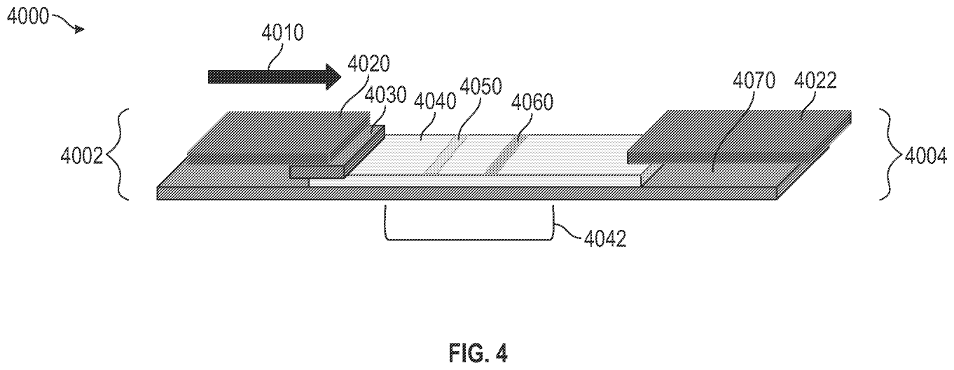

With reference to FIG. 4A, exemplary test strip device 4000 is shown. A proximate portion of device 4002 with a sample wicking pad 4020 is dipped into a urine sample such that urine sample flows up strip 4000 in a direction depicted by arrow 4010 and is stopped by adsorbent pad 4022 at a distal portion of device 4004 opposite sample pad 4020. Sample pad 4020 may be obtained from any commercially available source. In one embodiment, test strip 4000 includes conjugate pad 4030 that can be a glass fiber conjugate pad saturated with colloidal gold, colored latex beads, fluorescent dye, or other detectable labels that are conjugated to anti-LH beta antibodies of a first LH beta epitope. Test strip 4000 further comprises a membrane 4040. Membrane 4040 of test strip 4000 can be a nitrocellulose membrane with pore size between 3 to 20 μm. Membrane 4040 includes a detection zone 4042. A test line 4050 and a control line 4060 are impregnated on membrane 4040 within detection zone 4042. Membrane 4040 is supported by a backing card 4070. In one embodiment, test line 4050 is comprised of anti-LH beta antibodies of a second LH beta epitope. In one embodiment, control line 4060 is comprised of anti-mouse antibody. In some embodiments, where control line 4060 does not appear, the test results are invalid.

Test strip device 4000 using LH beta can be compared to typical Intact LH test. In an Intact LH test, the conjugate pad is saturated with LH alpha antibody conjugated to a colloidal gold, colored latex beads, fluorescent dye, or other detectable labels while the test line is impregnated with LH beta antibody. In some embodiments, the conjugate pad is saturated with LH beta antibody conjugated to a colloidal gold, colored latex beads, fluorescent dye, or other detectable labels while the test line is impregnated with LH alpha antibody.

In an embodiment, test strip 4000 is configured to comprise an anti-LH beta antibody conjugated to a detectable label at a concentration of a value selected from within the range of 15-45 mIU/mL within conjugate pad 4030. In an embodiment, test strip 4000 is configured to comprise an anti-LH beta antibody conjugated to a detectable label at a concentration of a value selected from within the range of 20-25 mIU/mL within conjugate pad 4030. In an embodiment, test strip 4000 is configured to comprise an anti-LH beta antibody conjugated to a detectable label at a concentration of 20 mIU/mL within at least one conjugate pad. In various embodiments, the detectable label includes colloidal gold. In various embodiments, the colloidal gold particles chosen to make up the anti-LH beta antibody-colloidal gold conjugate are of a size dimension of a value selected from the range of 20-100 nm. In various embodiments, the colloidal gold particles chosen to make up the anti-LH beta antibody-colloidal gold conjugate are of a concentration of 0.7-1.3 OD. The present inventor has recognized that the application of the anti-LH beta conjugated to a detectable label at the concentration levels described above, when applied to conjugate pad of the test strip 4000 in conjunction with the application of the LH beta-carrier protein conjugate of the specific concentration levels described herein applied to test line 4050 of test strip 4000, accomplishes the proper ratio of those specific binding partners to enable the test strip to detect for the presence of LH beta in a sample of urine applied to test strip 4000, and further visually indicate that the sample of urine contains LH beta above the pre-defined threshold or detectably indicate that the sample of urine does not contain LH beta above the pre-defined threshold.

Therefore, in various embodiments, the present inventor has recognized that the test strip configured as described effectively and reproducibly produces a negative test result if the LH beta level in tested urine is below approximately 20 mIU/mL; and the test strip configured as described effectively and reproducibly produces a positive test result if the LH beta level in tested urine is above approximately 20 mIU/mL. The present inventor recognizes that due to variations in the ability of different populations to metabolize progesterone in urine, the 20 mIU/mL threshold is not always the appropriate threshold. Therefore, various embodiments of test strip 4000 are configured to display a positive or negative result based on pre-defined thresholds of a LH beta level of a value selected from values within the range of 15 mIU/mL and 45 mIU/mL or, optionally within the range of 20 mIU/mL and 25 mIU/mL. The present inventor has recognized that embodiments of the invention are appropriately used in association with tracking LH beta levels during ovulation, since as ovulation progresses, LH beta levels also increase. Therefore, having a predetermined threshold of a higher value (e.g. 15 mIU/mL and 45 mIU/mL, or 20 mIU/ml and 25 mIU/mL) allows the user to monitor LH beta levels through the duration of pregnancy. Such monitoring, with strips configured to measure LH beta with a pre-defined threshold from within the range of 15 mIU/mL and 45 mIU/mL, or 20 mIU/mL and 25 mIU/mL, allows the user to confirm that LH beta levels are increasing appropriately throughout ovulation.

In some embodiments, the devices, systems and methods described herein make use of a conjugate comprising an antibody bound to a detectable label. In some variations, the detectable label may be colored particles, such as a metal sol or colloid. For example, in one variation, the detectable label comprises gold or latex beads, soluble dyes, or fluorescent dyes).

Any detectable label recognized in the art as being useful in various assays could be used in the devices, systems and methods herein. In particular, the detectable label can include compositions detectable by spectroscopic, photochemical, bio-chemical, immunochemical, or chemical means. As such, the detectable label produces a detectable signal. For instance, suitable labels include soluble dyes, fluorescent dyes, chemi-luminescent compounds, radioisotopes, electron-dense reagents, enzymes, colored particles, or dioxigenin. The detectable label can generate a measurable signal, such as radio-activity, fluorescent light, color, or enzyme activity, which can be used to identify and quantify the amount of label bound to a test line. Thus, the detectable label can also represent the presence or absence of a particular antigen bound thereto.

In certain embodiments, the detectable label comprises a gold colloid having a mean particle size of about 30 nm to about 80 nm prior to formation of the labeled conjugate. In one embodiment, the mean particle size can range from about 40 nm to about 60 nm prior to formation of the labeled conjugate.

Diagnostic tests for screening analytes, e.g. urinary hormones or metabolites thereof, may utilize antibodies specific to the analyte. A change in the level from a predetermined threshold level may be noted by differences in color or color intensity compared with the color in a reference window or reference guide. The color change may be produced using techniques such as enzyme-linked immunosorbent assays or lateral flow color matching assays to indicate the amount of analyte in a urine sample.

In some aspects, provided is a machine, optionally a base unit or digital reader configured to detect an indication by a test strip device as described herein. In some embodiments, the indication by the test strip of the onset of ovulation is presented by a detectable label. In certain embodiments the detectable label is perceptible by the human eye. In other embodiments, the detectable label includes fluorescent dye imperceptible to the human eye readable only with the assistance of a base unit or digital reader.

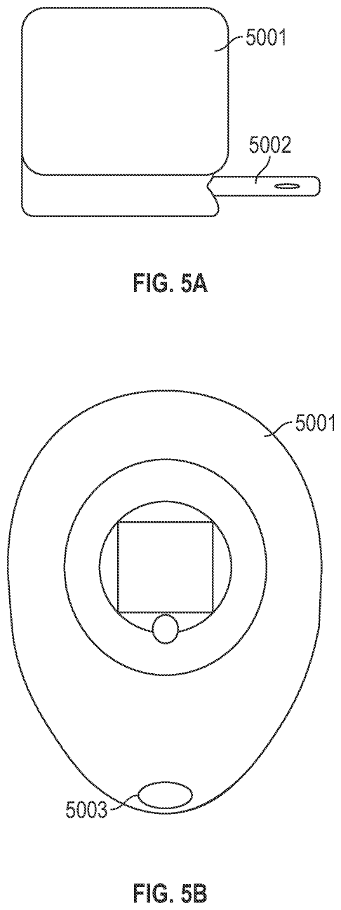

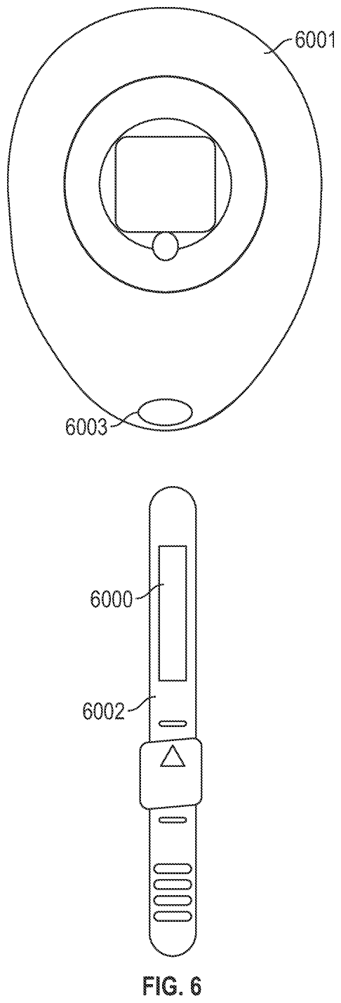

With reference to FIGS. 5A and 5B, exemplary base unit 5001 shown in an intended use in conjugation with a cartridge 50002 containing a test trip configured to evaluate a bodily fluid (e.g., urine sample) for the presence or absence of LH beta, is depicted. Base unit 5001 comprises a port 5003 for receiving cartridge 5002. With reference to FIG. 6, an embodiment of test strip 6000 contained within cartridge 60002 in exemplary use in association with base unit 6001 is depicted. Base unit 6001 comprises a port 6003 for receiving cartridge 600. In various aspects of the systems and methods herein, test strip 6000 configured for utilization in conjunction with base unit 6001, optionally configured to evaluate test strip 6000 contained within cartridge 6002, together comprise a diagnostic test system.

In some variations, the device described herein employs a sandwich technique, and the antibody used in the detection comprises a binding member or site which binds to an epitope on the analyte, hormone or hormone component for detection, such as LH beta. In certain variations, the antibody may have a detectable label bound thereto to provide a labeled antibody. The labeled antibody reacts with the analyte to form a complex in the non-serum bodily fluid. The analyte, which is bound with the labeled antibody, reacts with a capture antibody to form a “sandwich” comprising the capture antibody, analyte, and the labeled antibody. This sandwich complex is progressively produced as the test liquid with the analyte therein continuously moves along the test strip of the device. As more and more analyte/labeled antibody complex is immobilized as a sandwich with the capture antibody at the test line, the detectable labels aggregate and become visible through a viewing window, indicating the presence of a particular analyte in the non-serum bodily fluid. Embodiments can include one or more standards or internal controls that allow for determination of whether signal development (e.g., color development) is a true indication of the presence or absence of analyte, hormone or hormone component (e.g., LH beta) in the sample or is simply an artifact, such as caused by nonspecific sorption.

Generally, a capture antibody is an antibody, such as a monoclonal or polyclonal antibody, attached to a substrate directly or indirectly, such as a solid substrate. In some variations, the capture antibody includes at least one binding member that specifically or preferentially binds a particular, distinct epitope of an antigen, such as LH Beta or Intact LH.

In some embodiments, the device described herein employs a competitive assay technique for PDG detection. In one embodiment, the pre-defined threshold of PDG is determined by a fixed amount of PDG antibody on the conjugate pad and the amount of PDG conjugate impregnated on the membrane in competitive assay form. In some variations, the pre-defined threshold of LH beta is determined by a fixed amount of LH beta antibody on the conjugate pad and the amount of LH beta antibody on the membrane in sandwich assay form. In other embodiments, the sandwich assay form and the competitive assay form are integrated together into a single test. In one embodiment, the conjugate pad containing the LH antibody and the PDG antibody, and optionally antibodies of other hormones or analytes including FSH, Estrogen or estrogen metabolite, and HCG, are incorporated into a single conjugate pad within a single test. In an alternative embodiment, the conjugate pads containing the LH antibody and the PDG antibody, and optionally antibodies of other hormones or analytes including FSH, estrogen analyte, and HCG, are incorporated into at least two discrete conjugate pads within a single test.

In embodiments where the device is configured to detect LH beta using a sandwich technique, a pair of antibodies that binds to two unique surfaces of LH beta is desired. In certain variations of the foregoing, the pair of antibodies binds to different epitopes of LH beta. In one variation of the foregoing, the pair of antibodies binds to two unique binding surfaces on the same protein.

In other embodiments, the device is configured to detect LH beta using a competitive assay technique. In certain variations of the foregoing, the binding member is a single LH beta antibody. In one variation where a single LH beta antibody is used in the competitive assay, the antibody to LH beta is conjugated to the colloidal gold and placed on the conjugate pad. LH beta antigen is bound to a carrier protein, such as BSA, BGG, or OVA, and striped on the membrane's testing zone. When urine is applied, the LH beta in the urine binds the antibody-gold complex and moves past the testing zone. Any unbound antibody will bind to the test line, meaning that when LH in the urine sample is low, the line will be dark and when LH beta in the sample is high, the test line will be lighter. Therefore, the devices and methods described herein may employ different configurations to test for LH beta in a urine sample.

In some aspects, provided is a device configured to simultaneously analyze urine beyond mere analysis for the presence of LH beta, by further analyzing up to six analytes and/or hormones in an alternative test strip configuration featuring a test line configured to evaluate the non-serum bodily fluid for the presence of LH beta beyond a specific threshold and optionally additional test lines. In an embodiment, the test strip comprises a test line configured to evaluate urine for the presence of LH beta beyond a specific threshold, and at least a second test line. It is a teaching of an embodiment of the present invention for the test strip to optionally incorporate one or more additional test lines beyond the test line configured to analyze urine for the presence of LH beta, each additional test line specifically configured to evaluate urine for the presence of an item selected from the group consisting of progesterone or progesterone metabolites, such as PDG, Intact LH, HCG, FSH, testosterone and/or estrogen or analytes thereof, such as E1G or E3G. In some embodiments, the device comprises more than one test line, configured to detect for the presence of LH beta beyond a specific threshold and two additional test lines configured to detect for the presence of other analytes and/or hormones each beyond a specific threshold, in addition to a control line. In some embodiments, which can be particularly useful, the ovulation test device comprises a dual conjugate pad configured to hold two or more labeled antibodies.

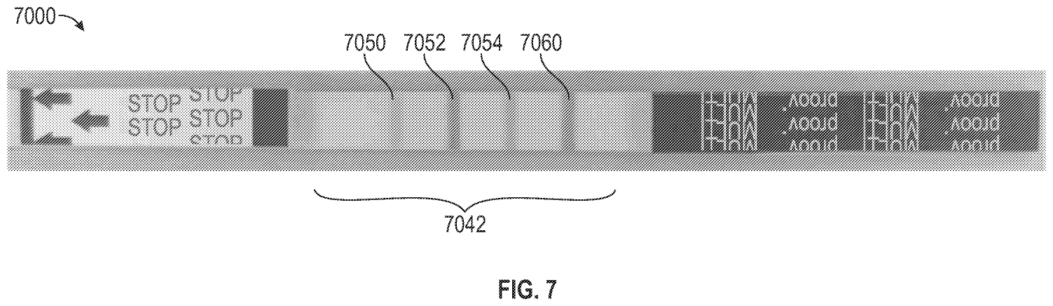

With reference to FIG. 7, test strip device 7000 configured to detect the presence of LH beta, estrone-3-glucoronide (also known in the art as E1G or E3G) and PDG in a single urine sample, is shown. In an embodiment, test strip 7000 includes detection zone 7042 comprising test line 7050 configured to evaluate urine for the presence of LH beta beyond a specific threshold, second test line 7052 configured to evaluate urine for the presence of PDG beyond a specific threshold third test line 7054 configured to evaluate urine for the presence of E1G beyond a specific threshold, and a control line 7060. Test strip 7000 may optionally incorporate one or more additional test lines beyond the test line configured to analyze urine for the presence of LH beta, each additional test line specifically configured to evaluate urine for the presence of an item selected from the group consisting of LH, HCG, FSH, testosterone and/or estrogen or analytes thereof, such as E3G.

In one embodiment, test strip 7000 is configured to simultaneously indicate, in a detection zone 7042, a positive or negative result for the presence of LH beta at test line 7050 above a pre-set threshold in a sample of urine applied to test strip 7000, in addition to indicating, at second test line 7052, a positive or negative result for the presence of at least one additional analyte and/or hormone, and, indicating at additional third test line 7054, a positive or negative result for the presence of at least one additional analyte and/or hormone, all contained within a single test strip 7000. In an embodiment, the at least one additional analyte and/or hormone to be tested at second test line 7052 and the at least one additional analyte and/or hormone to be tested at third test line 7054 is selected from the following group: (1), Estradiol (E2) or Estrogen metabolites (e.g. E1G or E3G) at concentrations 0-400 ng/ml in a competitive assay format; (2), Follicle Stimulating Hormone (FSH) at concentrations 3-40 mIU/ml in a sandwich assay format; (3), Luteinizing Hormone (LH) at concentrations 0-25 mIU/ml in a sandwich assay format; (4), Progesterone (P4) at concentrations of 0-40 ng/ml or progesterone metabolite (PDG) at concentrations of 3-20 μg/mL in a competitive or sandwich assay format; (5) human chorionic gonadotropin, (HCB) at concentrations of 0-10,000 mIU/ml; and (6) Testosterone, at concentrations of 0 to 50 μg, in a sandwich assay format. In an embodiment, the analyte and/or hormone that the test strip 7000 will simultaneously measure at second test line 7052 and optionally at third test line 7054 are selected from the group consisting of E2, E1G (or E3G), FSH, Intact LH, P4, PDG, Testosterone and HCG. In an embodiment, the second test line 7052 is configured to detect for the presence of a hormone or analyte differing from the hormone or analyte detected by the third test line 7054. In an embodiment, a digital reader is configured to evaluate the second test line and optionally the third test line in association with the methods further described herein. In an embodiment, the test strip further incorporates, in addition to a test line, a second test line, and a third test line, a fourth test line configured to similarly detect for the presence of a hormone or analyte differing from the hormone or analyte detected by the other (first, second and third) test lines within the test strip. The methods of evaluation and further configurations optionally applied to test strip 7000 associated with the testing of analytes and/or hormones beyond and in addition to LH beta are further described in U.S. patent application Ser. No. 15/974,229, which is hereby incorporated by reference in its entirety.

In an embodiment, labels (such as colloidal gold) are varied, with a separate and distinct label configured to attach to a separate hormone. In such embodiments, the present inventor has recognized the advantage that the test strip is configured to provide a different color for each hormone analyte indicating either the presence or absence of each hormone analyte following application of urine to the test strip. In an embodiment, the test strip is configured to comprise a conjugate pad (the conjugate pad also referred to as the “receiving zone” herein) comprising anti-LH antibody-colloidal gold conjugate, and at least one other conjugate. In an embodiment, the at least one other conjugate comprises anti-PDG antibody conjugated with a different label, optionally differently colored latex beads.

The present inventor has recognized that LH beta and HCG commonly exhibit cross reactivity, specifically due to the fact that HCG can bind to LH beta antibodies. Therefore, having different colors corresponding to the presence of different hormones provides a benefit by allowing an observer to determine whether cross-reactivity has taken place. In other words, if an area designated to test for the presence of LH beta displayed the coloration of the label for HCG, one skilled in the art would understand such a read to indicate that cross-reactivity has been demonstrated and that the test therefore is invalid. Alternatively, the present inventor has noted that due to the similarities in structure between estrogen analytes and progesterone analytes (in at least one example, said progesterone analytes consisting of PDG), cross reactivity could take place between those two hormone metabolites specifically. Thus, it is beneficial to have different hormones or hormone metabolites labelled with different colors. Such labelling is accomplished in an embodiment by binding to colloidal gold and/or one or more differently colored latex beads, each test line within the strip featuring a differently colored label, and is therefore a teaching of an embodiment of the invention.

In some variations, the test strip relies on the certain reagents being able to interact with other reagents to produce color in the test line of the membrane. Specifically, in the absence of LH beta hormone in the urine sample, the following reagents must interact in order for the test results to be useful. First, in one embodiment, colloidal gold must be conjugated to the immunologically active anti-LH beta antibody. In alternative embodiments, as a replacement for colloidal gold in other embodiments described herein, an alternative visual dye such as latex beads may be utilized to a similar effect. Further, in some embodiments, the colloidal gold conjugated anti-LH beta antibody must interact with the binding member that exhibits a moderate to high affinity for LH beta. Moreover, the binding member that exhibits moderate to high affinity for LH beta must bind a nitrocellulose membrane. The present inventor has recognized that for these embodiments to function as intended, these interactions between and among the colloidal gold conjugated anti-LH beta antibody and the binding member that exhibits a moderate to high affinity for LH beta, must be strong enough and stable enough to form and stay bound during urine sample application and lateral flow of the fluid across the reaction zone to solve the problems faced by the suboptimal prior art mechanisms described elsewhere herein. The disclosures in this paragraph constitute teachings of an embodiment of the invention.

In certain embodiments, the device can include a positive control. Thus, when exploiting the sandwich technique for example, a cell may have an authentic sample of the analyte for detection immobilized at a control line. If no color develops at this control line, the assay is considered inconclusive. When exploiting the competitive technique, the development of color at the positive control line means the assay results are inconclusive.

In yet another embodiment, which can be particularly useful when the ovulation test device comprises a control line disposed on the membrane downstream of the test line. The control line has immobilized thereon at least one capture antibody (e.g., a protein). The primary function of the control line is to capture and immobilize labeled antibody which has not been captured at the test line.

In some embodiments, the control line can include polyclonal antisera specific for the labeled antibody immobilized thereon. Indication of the presence of the detectable label at the control line indicates proper functioning of the test, irrespective of the presence or absence of analyte, hormone or hormone component in the sample. In some variations, both the test and control lines are visible on the test device to the user.

In association with embodiments herein, conjugate pad and membrane are joined together to form a single liquid path in association with a lateral flow assay. Reagents for detecting, labeling, and capturing an analyte of interest are disposed on the conjugate pad and membrane. Located on the conjugate pad is a binding member reactive with a first epitope of the analyte of interest, for example LH beta, Intact LH, FSH, P4, HCG and pregnanediol. The labeled conjugate further comprises a detectable marker (or label), such as colloidal gold. The labeled conjugate is bound to the conjugate pad such that when the solvent front created by the non-serum bodily fluid being analyzed passes through the conjugate pad, the labeled conjugate become solubilized by the liquid and flow with the solvent along the liquid path. In operation, if any analyte is present in the non-serum bodily fluid, it reacts with the labeled conjugate, then advances along the liquid path to form a sandwich complex when it reaches the test line of the membrane, where a sandwich complex is formed.

In some embodiments, the membrane comprises the other member of the affinity pair specific for a capturable component. The affinity member is immobilized, such as by simple adsorption, at the test line, and does not advance with the solvent front. In one embodiment, a control line also is located on the membrane downstream of the test line. The control line has immobilized thereon a binding agent having an affinity for the labeled binding member. The binding agent will capture any labeled binding member which is not captured at the upstream test line. In operation, the presence of the detectable marker at the control line indicates that sorptive transport has operated properly.

In operation, if any analyte is present in the liquid sample, it reacts first with the labeled binding member, then with the capturable component as the front advances along the liquid path. By the time the solvent front reaches the membrane section of the biphasic material, the capturable complex has formed. Polyclonal antisera and monoclonal antibodies or fractions thereof having specific binding properties and high affinity for virtually any antigenic substance which are useful as binding members and capture materials are known and commercially available, or can be produced from stable cell lines using well known cell fusion and screening techniques. The literature is replete with protocols for producing and immobilizing proteins. See, for example, Laboratory Techniques in Biochemistry and Molecular Biology, Tijssen, Vol. 15, Practice and Theory of Enzyme immunoassays, chapter 13, The Immobilization of immunoreactants on Solid Phases, pp. 297-328, and the references cited therein. The methods described herein also may be designed to exploit conventional “sandwich” or “competitive” techniques. In the case of the sandwich technique, the labeled binding member comprises an antibody which binds to an epitope on the analyte of interest to form a labeled antibody-antigen complex. This complex then migrates to the test line to react with a capturable component which, in this embodiment, comprises a second antibody specific for a second epitope of said analyte.

At the test line, the analyte and labeled antibody reacts with the immobilized capture member to form a “sandwich” of the second antibody, analyte and labeled antibody. This sandwich complex is progressively produced at the test line as sample continuously passes by. As more and more labeled conjugate is immobilized at the test line, the colored particles aggregate and become visible through the window of the casing, indicating the presence of the analyte in the liquid sample. Both in the presence or absence of a detectable level of analyte, the colored particles gather at the control line which also is visible through the window. In the case of the competitive technique, a known amount of the analyte (or hormone) of interest is present on the conjugate pad disposed upstream of an antibody specific for it. The analyte present in the conjugate pad is labeled. The labeled analyte on the conjugate pad may comprise, for example, an authentic sample of the analyte, or a fraction thereof which has comparable affinity for the antibody. As the liquid sample is transported along the conjugate pad, the labeled analyte present on the conjugate pad and any unlabeled analyte present in the sample compete for sites of attachment to the antibody. If no analyte is present in the sample, labeled analyte aggregates at the test line, and the presence of color indicates the absence of detectable levels of analyte in the sample. If analyte is present, the amount of labeled analyte which binds at the test site is reduced because of binding of analyte in the sample with the antibody, and no color, or a paler color, develops.

In some embodiments, the immunoassay device described herein is designed to detect human pregnancy. In one embodiment, the labeled binding member is a monoclonal antibody (MAb) against human chorionic gonadotropin (HCG) labeled with colloidal gold. For this purpose, MAb designated 2G9 may be used. Anti-HCG antibodies labeled with biotin are used for the capturable complex in an embodiment. Monoclonal antibodies which can be used for this purpose include the HCG specific monoclonal antibodies designated 2B2 and B 109 and CCF0 1. Methods for conjugating biotin to antibodies are well-known and do not form a part of the present invention. In one embodiment, the test line comprises streptavidin, which has a high affinity for biotin. A control line is located downstream of the test line. The control line has immobilized thereon goat anti-mouse IgG specific for the labeled anti-HCG. In another embodiment, the present immunoassay device is designed to detect human ovulation. In this embodiment, the labeled binding member comprises MAb 2G9, which is specific for luteinizing hormone (LH) and HCG, labeled with colloidal gold. The capturable complex comprises biotinylated LR-specific MAb LH26. The test line may comprise streptavidin and the control line comprises goat anti-mouse IgG specific for the labeled MAb. Further, in various embodiments the device may similarly assay other analytes such as, FSH and progesterone, estrogen or metabolites thereof.

In various embodiments of the invention, the test device comprises at least one specially configured conjugate pad. In an embodiment, the conjugate pad serves to receive a bodily fluid sample which may contain the metabolite of interest and to begin the flow of the sample along the test device. The conjugate pad is prepared from a natural or synthetic porous or macroporous material which is capable of conducting lateral flow of the fluid sample. A porous or macroporous material suitable for purposes of this invention generally has a pore size greater than 12 μm. Examples of porous materials include, but are not limited to, glass, cotton, cellulose, nitrocellulose, polyester, rayon, nylon, polyethersulfone, and polyethylene.

The conjugate pad can be formed from a substance which allows for release of indicator reagents. In certain embodiments, the conjugate pad comprises a bibulous, hydrophilic material, such as absorbent materials. In some variations, materials for use as a conjugate pad include, for example, cotton linter, cellulosic materials, or materials made of cellulose together with a polymeric fibrous material, such as polyamide or rayon fibers, and glass fiber material. The primary function of the conjugate pad is first to support and to subsequently release and transport various immunological components of the assay, such as a labeled conjugate capable of binding to the analyte of interest. This release and transport occurs during routine operation of the assay. Generally, the conjugate pad can be formed of any material capable of performing the function of holding, releasing, and transporting various immunological parts of the test such as the labeled test component.

In some variations, the materials useful in forming the conjugate pad may include: cotton linter paper, such as S&S 903, S&S GB002, and BFC 180 (available from Whatman, Fairfield, N.J.); cellulosic materials, such as Grade 939 made of cellulose with polyamide, Grade 989 made of cellulose blend fiber, and Grade 1278 and Grade 1281 made of cellulose and rayon with polyamide (available from Ahl-30 strom Corporation, Mt. Holly Springs, Pa.); and glass fiber, such as Lydall borosilicate (available from Lydall, Inc., Roch-ester, N.H.). The conjugate pad is coated with an aqueous solution containing bovine serum albumin (BSA) and a nonionic surfactant, such as Triton X-100, in order to prevent nonspecific binding and facilitate release of the diffusible reagents.

The membrane can be formed from a substance which permits immobilization of reagents for detection of the presence of analyte in the test fluid. In some variations, the membrane includes hydrophilic polymeric materials, such as microporous films or membranes, which permit protein reagents to be immobilized directly on the membrane by passive adsorption without the need for chemical or physical fixation. The devices, systems and methods described herein may use of chemical or physical fixation is, and any known method for immobilizing the reagents to the membrane may also be used.

In some variations, examples of materials useful as the membrane include a microporous polymeric film of nitrocellulose, nylon (e.g., nylon 66), or similar materials, or combinations of such materials. Materials for use as the membrane may have a pore size in the range of from about 5 μm to about 20 μm. In specific embodiments, the nitrocellulose membrane may be nitrocellulose alone or a mixed ester of nitrocellulose, such as in combination with an ester of nitric acid and/or other acids. The nitrocellulose membrane may be coated or laminated onto a translucent or transparent polymeric film to provide physical support for the membrane.

In one embodiment, a nitrocellulose polymer which has been cast onto a polyester film, such as MYLAR®, is used. Alternatively, a nitrocellulose membrane laminated onto a polyester film also may be used, although other backing materials besides polyester may be used. Pre-laminated or pre-cast sheets may be obtained from any commercially available source.

The diffusible and non-diffusible reagents can be applied to the conjugate pad and membrane, respectively, by any suitable technique. In one embodiment, the diffusible reagents are applied to the conjugate pad by direct application onto the surface of the medium and dried to form a narrow band.

In certain embodiments, provided are devices in which detection is based on a ratiometric analysis. Such embodiments can include a mixture of binding members comprising a first group of binding members that are selectively or preferentially reactive with an epitope of Intact LH and a second group of binding members that exhibit a moderate to high affinity for LH beta and are selectively or preferentially reactive with an epitope of LH beta. In certain embodiments, the first group of binding members that are selectively or preferentially reactive with an epitope of Intact LH are located at a different location than the second group of binding members that exhibit a moderate to high affinity for LH eta and are selectively or preferentially reactive with an epitope of LH beta. That is, each group of binding members can be striped independently of each other if desired. In one embodiment, the binding members that are selectively or preferentially reactive with LH beta comprise greater than 50% of the total number of binding members present in the mixture.

In various embodiments, the conjugate pad includes different groups of binding antibodies (e.g., pooled together in a single stripe, striped independently of each other, or striped on top or each other) including a first group of binding members that are selectively or preferentially reactive with an epitope of Intact LH and a second group that exhibits a moderate to high affinity for LH beta and are selectively or preferentially reactive with an epitope of LH beta. Alternatively, the test line can include different groups of capture antibodies including a first group preferably (but not necessarily) selectively or preferentially reactive with an epitope of Intact LH and a second group that exhibits a moderate to high affinity for LH beta and are selectively or preferably reactive with an epitope of LH beta. If desired, both the conjugate pad and the membrane can include the different groups of such antibodies (e.g., pooled together in a single stripe, striped independently of each other, or striped on top of each other).

In one embodiment, the test line comprises a mixture of Intact LH and LH beta specific antibodies, wherein about 50% to about 98% of the antibodies are selectively or preferentially reactive with LH beta and the remainder are selectively or preferentially reactive with Intact LH. Alternatively, the test line can comprise about 70% to about 95% of capture antibodies being selectively or preferentially reactive with LH beta, or from about 85% to about 95%, or from about 90% to about 95%. In one alternative embodiment, the test line comprises about 90% to about 99% of capture antibodies selectively or preferentially reactive with LH beta. In some embodiments, the LH beta antibody or antibodies also exhibit a moderate to high affinity for LH beta.

In another aspect, provided is an ovulation test device for selectively detecting LH beta in a non-serum bodily fluid which would allow multiple results to be conveyed to the consumer in one test kit by way of a multi-strip assay format. In some embodiments, the test device is a multi-line assay device for selectively or preferentially detecting LH beta in a non-serum bodily fluid deposited on a proximal portion of the device for transport to a distal portion of the device. In some embodiments, the device comprises a plurality of lateral flow test strips, wherein each lateral flow test strip further comprises a conjugate pad formed of a first material having a detectable label thereon. In some embodiments, the device comprises a membrane in fluid communication with the conjugate pad and formed of a second, different material, wherein the membrane comprises a test line. In some embodiments, at least one of the conjugate pad and the membrane of a first lateral flow test strip comprises a binding member that exhibits a moderate to high affinity for LH beta, and is selectively or preferentially reactive with LH beta. In some embodiments, at least one of the conjugate pad and the membrane of a second lateral flow test strip comprises a binding member that exhibits a moderate to high affinity for progesterone or progesterone metabolites, progesterone glucuronide (PDG), estrogen or estrogen metabolites, estrone-3 glucuronide (E1G), follicular stimulating hormone (FSH) or Intact LH, or any combination thereof, and is selectively or preferentially reactive with progesterone glucuronide (PDG), estrogen, estrone-3 glucuronide (E1G), follicular stimulating hormone (FSH) or Intact LH, or any combination thereof. In such embodiments, two or more independent test strips share a common fluid path to convey independent results.

The present inventor has discovered that because PDG is a small hormone metabolite, in order to strongly bind to the surface of a membrane, PDG requires a strong carrier protein, which is a teaching of an embodiment of the invention. However, the present inventor has discovered that, for the disclosed devices and methods to function as intended, not only does the strong carrier protein need to bind the nitrocellulose membrane of the test strip, but the strong carrier protein also needs to bind the PDG and present it to the anti-PDG antibody, which is a teaching of an embodiment of the invention.

Such teachings as disclosed herein solve the challenges associated with suboptimal prior art teachings, which lacked the ideal combination of a strong carrier protein able to bind the PDG and present it to the anti-PDG antibody.

The present inventor has discovered a unique combination of specific elements to allow for the detection of pregnanediol glucuronide (PDG) formulated such as to enable the creation of a pregnanediol glucuronide (PDG) urine test. In one embodiment, Bovine Gamma Globulin (BGG) is conjugated with PDG and combined with a mouse anti-PDG antibody of IgG2b isotype binding partner. In an alternative embodiment of the invention Bovine Gamma Globulin (BGG) conjugated to PDG is combined with a mouse anti-PDG antibody of IgG 1, IgG I Kappa, IgG2a or IgG2c isotype. The present inventor has recognized that such a specific combination uniquely allows for colloidal gold to be conjugated to the anti-PDG antibody of one of the specific isotypes mentioned above, and for the colloidal gold conjugated anti-PDG antibody to interact with the PDG-BGG conjugate. Other combinations have been attempted, and have failed to allow the colloidal gold to function to produce the color needed to allow the test results to be viewable visually by the naked and untrained (layperson) eye. The present inventor has noted that the utilization of BGG conjugated to PDG allows for anti-PDG antibody, specifically of the IgG2b isotype, to bind in such a manner that colloidal gold is carried at a concentration sufficient for naked eye visualization. The present inventor notes that Globulins evidence the preferable binding ratio of 8-32 PDG antigens per carrier protein, which favor presentation of a visual result perceptible to the naked eye or to a reader. It is therefore a teaching of embodiments of the invention to comprise a carrier protein demonstrating the binding ratio of 8-32 PDG antigens per carrier protein. The present inventor has recognized the benefit associated with embodiments of the invention described herein that a PDG test may be producible allowing the results to be visually interpreted with the naked eye.

In association with teachings of the invention, the test strip is configured to comprise a conjugate of a carrier protein demonstrating the binding ratio of 8-32 PDG antigens per carrier protein, optionally a Globulin carrier protein, with PDG. Such PDG-carrier protein conjugate is combined with a mouse anti-PDG antibody of one the class of the IgG isotypes in an embodiment. The class of Ig isotypes includes IgG 1, IgG2b, IgG 1 Kappa, IgG2a or IgG2c isotype as contemplated in association with embodiments of the invention. The conjugation of a carrier protein to PDG, and the combination of the PDG-conjugated carrier protein with a mouse anti-PDG antibody of Ig isotype is accomplished in accord with general conjugation procedures as well-known by those skilled in the art.

It is a further teaching that the specifically chosen anti-PDG antibody needs to be monoclonal, due to the nature of the PDG antigen presentation on the PDG-carrier protein conjugate. In order for the embodiments of the invention to function as intended, the specifically chosen anti-PDG antibody must incorporate one of the following isotypes: IgG 1, IgG2a, IgG2b, or IgG2c. The present inventor has discovered that isotypes other than IgG 1, IgG2a, IgG2b, or IgG2c, including but not limited to IgM, IgS, and IgE anti-PDG antibody isotypes, remain unable to effectively bind the colloidal gold (or other visual label) and produce a strong enough color signal on the reaction zone due to their size and structure and are therefore excluded from the preferred embodiment of the invention. Since the colloidal gold must bind the Ig region of the anti-PDG antibody. The present inventor has discovered that the IgG 1, IgG2a, IgG2b, and IgG2c isotypes of the anti-PDG antibody sufficiently bind colloidal gold and are therefore incorporated into embodiments of the invention. As a result, the IgG 1, IgG2a, IgG2b and IgG2c isotypes of the anti-PDG antibody therefore produce the strongest color. In one embodiment, the IgG2b isotype is included in the invention, as the present inventor has recognized that the IgG2b isotype performs slightly better when producing color. Therefore, certain embodiments of the invention incorporates the IgG2b isotype of the anti-PDG antibody. Alternative embodiments of the invention incorporate the IgG2a, IgG2c or IgG1 isotypes of the anti-PDG antibody.

The present inventor has recognized that the utilization of a Globulin within a specific combination uniquely allows for colloidal gold to be conjugated to the immunologically active anti-PDG antibody of one of the class of the IgG isotypes. In an embodiment, the combination enables the colloidal gold conjugated anti-PDG antibody to interact with the PDG-Globulin conjugate. In embodiments of the invention, therefore, the conjugate striped on the membrane in the testing area is PDG-Globulin and the anti-PDG antibody must be a monoclonal anti-PDG antibody of one of the following isotypes: IgG1, IgG2a, IgG2b, or IgG2c.

The present inventor recognizes that embodiments of the invention differ from other combinations that have been attempted in the prior art. Specifically, the other combinations have failed to allow the colloidal gold to function to produce the color needed to allow the test results to be viewable visually by the naked and untrained (layperson) eye. The present inventor has recognized that the novel utilization of a Globulin conjugated to PDG as described herein allows for anti-PDG antibody specifically of the Ig isotype, in accordance with the specific concentration levels described herein in embodiments of the invention, to bind in such a manner that colloidal gold is carried at a concentration sufficient for naked eye visualization. The present inventor has recognized the benefit associated with embodiments of the invention that a PDG test may be producible allowing the results to be visually interpreted with the naked eye and/or an external reader affordable to a typical consumer.

In some embodiments, one carrier protein is conjugated to eight or more PDG molecules. In one embodiment, the one carrier protein is conjugated to no more than thirty two PDG molecules. The present inventor has discovered that such a ratio allows for the colloidal gold conjugated anti-PDG antibody to bind with both enough affinity and avidity to produce a bright enough color in the test reaction zone for typical users to distinguish visually.

As referred to herein, a monoclonal anti-PDG antibody as described in the preceding paragraphs, and more specifically a monoclonal anti-PDG antibody having the necessary binding affinity for PDG such that when used in association with the invention as described herein it is capable of yielding a detection threshold of PDG of 3-20 μg/mL, has been deposited in accordance with the provisions of the Budapest Treaty at the American Type Culture Collection (ATCC), located at the following address: 10801 University Boulevard, Manassas, VA 20110 USA on Apr. 23, 2021. The accession number of the deposit is Patent Deposit Number PTA-127054. The deposited material is a biological material specifically identified in the application, namely a monoclonal anti-PDG antibody as specifically referred to herein, and more specifically an anti-PDG antibody as described in the preceding two paragraphs and elsewhere herein.

In certain embodiments, the detecting of the presence or absence of hormones and/or analytes comprises: (A) illuminating the first capture region (as used herein, the term “capture region” is also referred to as “test line”) and the second capture region with one or more light sources; (B) detecting the first optical signal from the first capture region and the second optical signal from the second capture region with one or more optical detectors; (C) determining an amount of the first analyte or hormone present in the biological sample based on a level of the first optical signal; and (D) determining an amount of the second analyte present in the biological sample (such as non-serum bodily fluid) based on a level of the second optical signal. In some embodiments, the first capture region is downstream of the second capture region on the test strip. In some embodiments, the second capture region is downstream of the first capture region on the test strip. In some embodiments, the first fluorescent label and the second fluorescent label are the same. In another aspect, an assay device is provided for determining a presence of at least a first analyte and a second analyte in a biological sample, the assay device comprising: a test strip defining a flow path and comprising: a) at a first end, a sample zone configured to be contacted with a biological sample suspected of containing the first analyte and the second analyte; b) a labeling zone having absorbed thereon a mobilizable first detection reagent conjugated to a first fluorescent label and a mobilizable second detection reagent conjugated to a second fluorescent label, which the first detection reagent specifically binds to the first analyte thereby forming a first analyte-first detection reagent complex and the second detection reagent specifically binds to the second analyte thereby forming a second analyte-second detection reagent complex; c) a capture zone comprising a first capture region and a second capture region, wherein the first capture region has immobilized thereon a first capture reagent which specifically binds to the first detection reagent when the first detection reagent is not in a complex with the first analyte, and the second capture region has immobilized thereon a second capture reagent which specifically binds to the second analyte-second detection reagent complex, wherein a first optical signal from the first fluorescent label is capable of being detected at the first capture region and which the first optical signal decreases with increasing amounts of the first analyte present in the biological sample, and wherein a second optical signal from the second fluorescent label is capable of being detected at the second capture region and which the second optical signal increases with increasing amounts of the second analyte present in the biological sample. In some embodiments, the first capture region is downstream of the second capture region on the test strip. In some embodiments, the second capture region is downstream of the first capture region on the test strip. In some embodiments, the assay device further comprises, downstream of the capture zone, a control zone comprising a first control region having immobilized thereon a first control reagent which binds to the first detection reagent and the second detection reagent, and a second control region having immobilized thereon a second control reagent which binds to the first detection reagent and the second detection reagent.

In some embodiments, the assay device is configured to be inserted into a reader device for detecting the first and second optical signals from the first and second fluorescent labels. In some embodiments, the assay device further comprises a readable, or readable and writable chip, configured to be read and wrote by the reader device. In some embodiments, the readable chip comprises information related to the biological sample, the assay device, or both. In some embodiments, the first capture reagent does not displace the first analyte from the first detection reagent if the first analyte is bound to the first detection reagent. In some embodiments, the first capture reagent does not bind to the first analyte-first detection reagent complex. In some embodiments, the second capture reagent does not bind to non-complexed second analyte or non-complexed second detection reagent. In some embodiments, the first fluorescent label and the second fluorescent label are the same. In some embodiments, the first fluorescent label and the second fluorescent label are different. In some embodiments, the first detection reagent, the second detection reagent, or both is an antibody or antibody fragment. In some embodiments, the first detection reagent, the second detection reagent, or both is an antigen. In some embodiments, the biological sample is blood, urine, or saliva. In some embodiments, the first analyte is PDG and the second analyte is luteinizing hormone (LH) or estrone-3-glucuronide (E3G). In some embodiments, the first detection reagent is an anti-PDG antibody and the second detection reagent is an anti-LH antibody or an anti-E3G antibody. In some embodiments, the first capture reagent is a protein-E3G antigen complex and the second capture reagent is an anti-LH antibody. In some embodiments, a decrease in the first optical signal and an increase in the second optical signal are indicative of a time of an elevated ovulation cycle of a mammal. In some embodiments, the first control reagent and the second control reagent are anti-mouse IgG antibody.

In one aspect, a diagnostic test system is provided comprising: a housing, comprising: a) a port for receiving an assay device, said assay device comprising two or more capture regions; b) a reader comprising: i) one or more light sources for illuminating said two or more capture regions; ii) one or more light detectors for detecting optical signals from said two or more capture regions; and c) a data analyzer having one or more processors configured to: A) receive said optical signals; and determine an amount of at least a first analyte and a second analyte present in a biological sample based on said optical signals, wherein an optical signal of a first of said two or more capture regions increases with decreasing amounts of said first analyte present in said biological sample, and an optical signal of a second of said two or more capture regions increases with increasing amounts of said second analyte present in said biological sample; wherein in an embodiment at least one of the said first capture region or the said second capture regions is configured to detect LH beta present in a biological sample in accord with at least the teachings herein.

In some embodiments, the diagnostic test system is further configured to detect optical signals from two or more control regions on the assay device. In some embodiments, the diagnostic test system is further configured to compare optical signals from the two or more control regions with optical signals from the two or more capture regions. In some embodiments, the assay device comprises any assay device described above. In some embodiments, the assay device further comprises a second end, configured to be inserted into the port of the diagnostic test system. In some embodiments, the data analyzer is configured to detect an optical pattern of the optical signals. In some embodiments, the optical pattern is a binary optical pattern. In some embodiments, the assay device further comprises a readable chip configured to be detected by the reader. In some embodiments, the readable chip comprises information related to the biological sample, the assay device, or both. In some embodiments, the reader is configured to transmit a data output to a mobile device. In some embodiments, the data output comprises a medical result based on the amount of first analyte and the amount of second analyte present in the biological sample. In some embodiments, the optical signals are fluorescent signals. The device may be used to test for the presence or absence of at least a first analyte and a second analyte in a sample. In some embodiments, the device may be used to determine an amount or a relative amount of at least a first and second analyte in a sample. Other features of the immunoassay device may include a test strip cassette for supporting and/or protecting the test strip. The cassette may be composed of a sturdy material such as plastic (e.g. high-impact polystyrene). The cassette may include, at a proximal end, a sample application window for applying a fluid sample to the sample pad. The cassette may further include an assay results window for visualization of the assay results. The assay results window may be positioned on the device directly above the capture zone and control zone such that a detectable signal can be visualized or read (e.g. by a diagnostic test system). The cassette may be of a certain size and shape so as to be compatible with a diagnostic test device of the disclosure, such that the cassette may be inserted into a cavity, port or receiver of a diagnostic test device. In one aspect, a diagnostic test system is provided comprising: a housing, comprising: a) a port for receiving an assay device, said assay device comprising two or more capture regions; b) a reader comprising: i) one or more light sources for illuminating said two or more capture regions; ii) one or more light detectors for detecting optical signals from said two or more capture regions; and c) a data analyzer having one or more processors configured to: A) receive said optical signals; and B) determine an amount of at least a first analyte and a second analyte present in a biological sample based on said optical signals, wherein an optical signal of a first of said two or more capture regions increases with decreasing amounts of said first analyte present in said biological sample, and an optical signal of a second of said two or more capture regions increases with increasing amounts of said second analyte present in said biological sample.

The diagnostic test system may include a housing for containing the components of the system. The housing can be constructed of any suitable material. The housing may be configured to receive an immunoassay device configured to detect for at least the presence or absence of LH beta of the disclosure. For example, the housing may include a port or opening for receiving the immunoassay device configured to detect for at least the presence or absence of LH beta. The cassette or housing of the immunoassay test device may include a cavity. The cavity, opening or port of the diagnostic test system may include a ball bearing contained within the inner wall of the chamber. The ball bearing may hook or latch into the cavity of the test device, thereby locking the immunoassay test device into the receiving chamber of the diagnostic test system.

The system may further include, contained within the housing, a reader device. The reader device may include one or more light sources for illuminating the immunoassay device or a region of the immunoassay device configured to detect for at least the presence or absence of LH beta. In one non-limiting example, the one or more light sources are configured to illuminate the detection zone of an immunoassay device configured to detect for at least the presence or absence of LH beta of the disclosure. The type of light source suitable for use with the immunoassay devices will depend on the chemistry of the immunoassay device. In one particular example, the one or more light sources are used to illuminate a detectable label provided by the immunoassay device configured to detect for at least the presence or absence of LH beta. In a particular example, the detectable label provided on the immunoassay device is a fluorophore, and therefore, the one or more light sources of the reader device should include a fluorescent light source (e.g., a light-emitting diode (LED)). It is to be understood that the wavelength of light provided by the light source of the reader device should be selected based on the excitation wavelength of the detectable label, and can readily be selected by a person of skill in the art.

The reader may be configured to illuminate the detection zone and/or the control zone of an immunoassay device configured to detect for at least the presence or absence of LH beta of the disclosure. For example, the reader may be configured to illuminate the first capture region configured to signal the presence or absence of LH beta, the second capture region, the first control region, the second control region, or any combination thereof. In some embodiments, the reader is configured to scan across the test strip of an immunoassay device. In such embodiments where the immunoassay device utilizes a single fluorophore, the reader may contain a single fluorescent light source. In embodiments where the immunoassay device utilizes more than one fluorophore, the reader may contain more than one fluorescent light source.

The reader may further comprise one or more light detectors (e.g., a photodetector) for detecting optical signals from the immunoassay device. Generally speaking, the one or more light detectors should be capable of distinguishing between emitted light at a first discrete position and a second discrete position on the immunoassay device. This may be accomplished by, e.g., the one or more light sources scanning across the test strip of the immunoassay device and determining the position of the emitted light on the immunoassay device.

The diagnostic test device may further comprise a data analyzer. The data analyzer may have one or more processors configured to receive an optical signal. In some embodiments, the data analyzer is in operable communication with a reader device. The data analyzer may be configured to determine an amount of analytes present in a sample, for example, by measuring an amount of optical signal produced at the detection zone of an immunoassay device configured to detect for at least the presence or absence of progesterone or a progesterone analyte. For example, the data analyzer may be configured to calculate the area under the curve of a signal intensity plot. The data analyzer may further be configured to determine the differences between signal intensities among the multiple discrete regions on the test strip. For example, the data analyzer may be configured to determine the difference between the signal intensity at the first capture region and the signal intensity at the second control region. The data analyzer may further be configured to determine the difference between the signal intensity at the second capture region and the signal intensity at the first control region. The data analyzer may further be configured to calculate an amount or concentration of the analytes present in the sample, in one aspect at least one of the analytes present being LH beta. The data analyzer may be further configured to detect a binary optical pattern. The binary optical pattern can be generated by two fluorescent materials which excitation and/or emission spectrum differs in wavelength. In some embodiments, the binary optical pattern can be generated by one fluorescent material and one light absorbent material. The detection reagents may be conjugated with the two types of materials respectively and can be captured in the same detection zone, such that the detection zone may generate two different optical signal patterns in the data analyzer.

In various aspects, the system may comprise a housing for containing the electronic components of the system such as that shown in FIGS. 5A, 5B and 6. The system may have a top housing and a bottom housing. The top housing may comprise a display module for displaying the results of an immunoassay configured to detect for at least the presence or absence of LH beta as described herein. The system may further comprise a display cover. The system may further comprise a battery. The system may further comprise an optomechanics module. The optomechanics module may comprise the one or more light sources and one or more light detectors as described above. The system may further comprise a circuit board containing electronic components. The cassette or housing of the immunoassay test device configured to detect for at least the presence or absence of LH beta may include a cavity. The chamber or receiving port of the diagnostic test system may include a ball bearing contained within the inner wall of the chamber. The ball bearing may hook or latch into the cavity of the test device, thereby locking the immunoassay test device configured to detect for at least the presence or absence of LH beta into the receiving chamber of the diagnostic test system.

The diagnostic test system may include an optomechanics module comprising the one or more light sources for illuminating the test strip of the immunoassay device configured to detect for at least the presence or absence of LH beta. The optomechanics module may be movable across an optical axis such that the optomechanics module moves laterally across the test strip of the immunoassay device configured to detect for at least the presence or absence of LH beta, thereby scanning the test strip. The diagnostic test system may further comprise an actuation module. The actuation module may comprise one or more motors configured to actuate/move the optomechanics module. In some embodiments, the motors may be coupled to a rack and pinion mechanism that is configured to translate the optomechanics module along one or more directions. For example, the optomechanics module can be translated along a longitudinal axis of the test strip of the immunoassay device configured to detect for at least the presence or absence of LH beta. The direction(s) of translation may or may not be orthogonal to an optical axis of the optomechanics module. The direction(s) of translation may be parallel to the longitudinal axis of the test strip, and the optical axis may be orthogonal to the longitudinal axis or a planar surface of the test strip. In some embodiments, the direction(s) of translation need not be parallel to the longitudinal axis of the test strip, and the optical axis need not be orthogonal to the longitudinal axis (or a planar surface) of the test strip. For example, the direction(s) of translation and/or the optical axis may be at an oblique angle relative to the longitudinal axis of the test strip.

In various aspects, the diagnostic test system may include an optical configuration suitable for use with the diagnostic test system and positioning of the optics above a test strip of an immunoassay device configured to detect for at least the presence or absence of LH beta. The optical configuration may include a light source (e.g., a light-emitting diode (LED) for illuminating the test strip. The optical configuration may further include one or more lens, a filter, optical beamsplitters, or any combination thereof. The optical configuration may further include a photodetector for detecting an optical signal from the immunoassay device configured to detect for at least the presence of LH beta. In an example, the system is configured to an excitation/emission spectra with an excitation wavelength of 492 nm and an emission wavelength of 512 nm.

In some embodiments, the diagnostic test device generates measurement results (e.g., concentration or relative amounts of analytes present in the sample) from a completed assay performed on the test device, as described throughout. In some embodiments, the diagnostic test device displays the measurement results on a screen contained within the device. Data containing the measurement results can be transmitted from the diagnostic test device to a mobile device and/or to a server. The data may be transmitted via one or more wireless or wired communication channels. The wireless communication channels may comprise Bluetooth, RTM, WiFi, RFID, near field communication, 3G, 4G, and/or 5G networks. The data containing the measurement results may be stored in a memory on the diagnostic test device when the diagnostic test device is not in operable communication with the mobile device and/or the server. The data may be transmitted from the diagnostic test device to the mobile device and/or the server when operable communication between the diagnostic test device and the mobile device and/or the server is re-established.

Further provided herein, in one aspect, for inclusion within the context of a system for evaluating the onset and quality of ovulation, are kits which may include any number of immunoassay test devices configured to detect for at least the presence of LH beta and/or reader devices of the disclosure. In one aspect, a kit is provided for determining qualitatively or quantitatively the presence of LH beta and a second analyte in a biological sample, the kit comprising: a) an assay device configured to detect for at least the presence of LH beta according to an embodiment of the disclosure; and b) instructions for using the kit.

### EXAMPLES

The presently disclosed subject matter will be better understood by reference to the following Examples, which are provided as exemplary of the invention, and not by way of limitation.

### Example 1

Comparing Daily LH Testing with LH Beta Vs. Intact LH

This example compares the use of an assay that detects LH beta with an assay that detects Intact LH to determine LH surge at a threshold, e.g., 20-25 mUI/ml.

The LH beta assay used in this example was a standard lateral flow format in which LH antibody MH3 was conjugated to colloidal gold, and was sprayed on the conjugate pad. The detection zone was the MH6 antibody striped on the membrane. Urine was applied so that the flow would free the gold/MH3 particles and they would pass over the MH6 antibody. If the samples contained LH, it would bind the MH3 gold and MH6 line and create a red color. The intensity of the red color is directly proportional to the amount of LH in the urine sample. It should be understood that, in other variations, MH3 and MH6 may be reversed and latex colored beads of fluorescent tag may be used as the visual label.

Daily first morning urine (first urine of the day after the longest sleep was collected and analyzed for LH and PDG levels. Urine was collected for 6 consecutive days. The same urine samples was used to measure LH in 3 different devices, the LH beta assay and 2 commercially available tests that track Intact LH (Proov Predict LH; Mira Fertility Plus Wands).

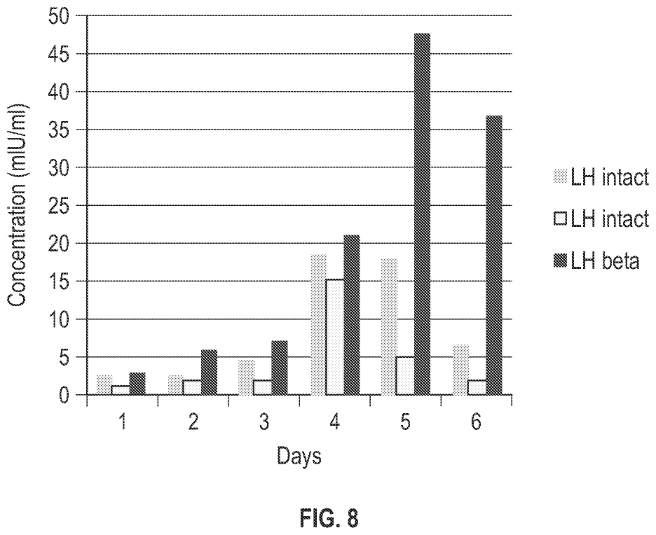

Results are shown on FIG. 8. All assays show a significant increase in LH levels on the fourth day of testing. Neither of the Intact LH tests were able to detect a level of LH over 20 mIU/ml, which is typically needed to show ovulation is near. On the other hand, the LH beta test showed a level of LH over 20 mIU/ml on the 4th, 5th and 6th days of testing. Ovulation was confirmed with an increase in PDG levels on the 6th day of testing. These results suggest that there is a high chance the LH surge will be missed if only testing once a day and measuring intact LH. An assay that detects LH beta gives the woman the ability to test LH once a day and not risk missing their fertile window. This example demonstrates that an assay that detects LH beta in the urine of a woman has a higher chance of seeing LH surge above a set threshold (such as, 20-25 mUI/ml).

1. A device for selectively or preferentially detecting LH beta in a non-serum bodily fluid deposited on a proximal portion of the device for transport to a distal portion of the device,
wherein the device comprises:
a conjugate pad formed of a first material having a detectable label thereon; and
a membrane in fluid communication with the conjugate pad and formed of a
second, different material, wherein the membrane comprises a test line, and
wherein at least one of the conjugate pad and the membrane comprises a binding member that exhibits a moderate to high affinity for LH beta, and is selectively or preferentially reactive with LH beta.

2. The device of claim 1, wherein the test line further comprises a binding member that exhibits a moderate to high affinity for Intact LH or is selectively or preferentially reactive with Intact LH.

3. The device of claim 1, further comprising one or more additional test lines selectively or preferentially reactive with other hormones or analytes.

4. The device of claim 3, wherein the other hormones or analytes are progesterone or a metabolite of progesterone, progesterone glucuronide (PDG), estrogen or estrogen metabolites, estrone-3 glucuronide (E1G), follicular stimulating hormone (FSH) or Intact LH, or any combination thereof.

5. The device of claim 3, wherein the one or more additional test lines are located anywhere between the conjugate pad and end of the detection zone.

6. The device of claim 1, wherein at least one of the conjugate pad and the membrane can include a binding member that is reactive with LH beta.

7. The device of claim 6, wherein the binding member is selectively or preferentially reactive with LH beta and exhibits a moderate to high affinity for LH beta.

8. The device of claim 1, further comprising a mixture of binding members.

9. The device of claim 8, wherein the mixture of binding members comprises:
a first group of binding members that are selectively or preferentially reactive with LH beta, or any part of LH beta, or LH alpha, or any part of LH alpha; and
a second group of binding members that exhibit a moderate to high affinity for LH beta, and are selectively or preferentially reactive with an epitope of LH beta.

10. The device of claim 1, wherein the binding member is the binding site of an antibody.

11. The device of claim 10, wherein the antibody is optionally bound to a detectable label.

12. The device of claim 11, wherein the detectable label comprises, colloidal gold, soluble dyes, fluorescent dyes, chemi-luminescent compounds, radioisotopes, electron-dense reagents, enzymes, colored particles, or dioxigenin.

13. The device of claim 1, wherein the device is configured to measure the amount of LH beta by the presence of a detectable label at the test line.

14. The device of claim 1, wherein the device is configured to detect the presence of LH beta by the presence of color development at the test line.

15. The device of claim 1, wherein the device further comprises a control line, wherein the control line is configured to indicate that a result is conclusive or inconclusive.

16. The device of claim 1, wherein the non-serum bodily fluid is a urine sample.

17. A device for selectively or preferentially detecting LH beta in a non-serum bodily fluid deposited on a proximal portion of the device for transport to a distal portion of the device, wherein the device comprises:
a plurality of lateral flow test strips, wherein each lateral flow test strip further comprises:
a conjugate pad formed of a first material having a detectable label thereon; and
a membrane in fluid communication with the conjugate pad and formed of a second, different material, wherein the membrane comprises a test line;
wherein at least one of the conjugate pad and the membrane of a first lateral flow test strip comprises a binding member that exhibits a moderate to high affinity for LH beta, and is selectively or preferentially reactive with LH beta; and
wherein at least one of the conjugate pad and the membrane of a second lateral flow test strip comprises a binding member that exhibits a moderate to high affinity for progesterone or progesterone metabolites, progesterone glucuronide (PDG), estrogen or estrogen metabolites, estrone-3 glucuronide (E1G), follicular stimulating hormone (FSH) or Intact LH, or any combination thereof, and is selectively or preferentially reactive with progesterone glucuronide (PDG), estrogen, estrone-3 glucuronide (E1G), follicular stimulating hormone (FSH) or Intact LH, or any combination thereof.

18. The device of claim 17, wherein the second lateral flow test strip comprises a binding member that exhibits a moderate to high affinity for progesterone glucuronide (PDG) and is selectively or preferentially reactive with progesterone glucuronide (PDG).

19. The device of claim 17, wherein the second lateral flow test strip comprises a binding member that exhibits a moderate to high affinity for Intact LH, and is selectively or preferentially reactive with Intact LH.

20. The device of claim 17, wherein the test strips share a common sampling component, wherein the common sampling component allows sample to be transferred to both strips simultaneously.

21. The device of claim 1, wherein the device evaluates for the presence of LH beta in a range between 15-40 mIU/mL.

22. The device of claim 1, wherein the device evaluates for the presence of LH beta in a range between 20-25 mIU/mL.

23. The device of claim 4, wherein the device evaluates for the presence of PDG in a range between 3-20 μg/mL.

24. The device of claim 1, wherein the device is configured to confirm an LH surge based on a single test taken to indicate the presence of LH beta at or above a pre-defined threshold.

25. The device of claim 24, wherein the single test is taken once daily until the first positive result for LH beta in the non-serum bodily fluid at or above the pre-defined threshold.

26. The device of claim 24, wherein the device allows early detection of an LH surge by detecting LH beta at an earlier date than the detection of Intact LH at or above the pre-defined threshold.

27. The device of claim 24, wherein LH beta is detected 2-3 days earlier than Intact LH.

28. The device of claim 24, wherein the predefined threshold indicative of the onset of ovulation is at or above 20 mIU/mL.

29. A method of evaluating the onset and quality of ovulation, comprising:
providing a device according to claim 1 for selectively or preferentially detecting LH beta;
applying a non-serum bodily fluid comprising (i) LH beta or (ii) LH beta and another hormone or analyte to the device; and
detecting the presence of LH beta, wherein the detected presence of LH beta indicates the onset of ovulation.

30. The method of claim 29, wherein the non-serum bodily fluid applied to the device comprises LH beta and PDG, and the method further comprises detecting the presence of PDG,
wherein the detected presence of LH beta indicates the onset of ovulation, and the detected presence of PDG on at least two of the days during the period of 7-10 days following the detected presence of LH beta indicates a high quality of ovulation.

31. The method of claim 30, wherein the method further comprises estimating the fertile window dates, optionally by repeating the providing, applying and detecting steps, the fertile window opening date estimated as the date of a positive result for the presence of LH beta and the fertile window closing date estimated as the date of a positive result for the presence of PDG.

32. The method of claim 29, wherein the non-serum bodily fluid is evaluated daily for the presence of LH beta, starting 3-15 days after the onset of menstruation.

33. The method of claim 29, wherein only the first positive result for LH beta indicated by a single positive test is needed to indicate the onset of ovulation.

34. The method of claim 29, wherein the method allows early detection of the onset of ovulation by detecting LH beta at or above a pre-defined threshold that indicates the onset of ovulation, at an earlier date than the detection of Intact LH at or above the pre-defined threshold that indicates the onset of ovulation.

35. The method of claim 34, wherein LH beta is detected 2-3 days earlier than Intact LH.

36. The method of claim 34, wherein only once daily testing is required to detect LH beta at a concentration at or above the pre-defined threshold that indicates the onset of ovulation.

37. The method of claim 34, wherein the predefined threshold that indicates the onset of ovulation is at or above 20 mIU/mL.
